(19) United States
(12) Patent Application Publication
(10) Pub. No.: US 2016/0120864 A1
(43) Pub. Date: May 5, 2016

(54) Novel Pyrrole Derivatives

(71) Applicant: UNIVERSITY OF LEICESTER, Leicester (GB)

(72) Inventors: Peter William ANDREW, Leicester (GB); Mafalda Pires DAMASO, Leicester (GB); Mark William DAVIES, Nottingham (GB); Fritz-Frieder FRICKEL, (US); Rana LONNEN, Leicester (GB); Daniel HAMZA, Nothingham (GB); Simon Christopher HIRST, Nottingham (GB)

(21) Appl. No.: 14/896,333

(22) PCT Filed: Jun. 4, 2014
(86) PCT No.: PCT/GB2014/051725

§ 371 Date: Dec. 4, 2015

(30) Foreign Application Priority Data
Jun. 4, 2013 (GB) 1309935.3

(51) Int. Cl. A61K 31/496; A61K 45/06; C07D 207/36

(52) U.S. Cl. CPC A61K 31/496; C07D 207/36; A61K 45/06

(57) ABSTRACT

There is provided inter alia novel N-phenyl substituted pyrrole derivatives and their use in therapy, especially in the treatment of bacterial (e.g. pneumococcal) infections.

### FIELD OF THE INVENTION

The invention relates to compounds which are cytolysin inhibitors, and prodrugs thereof, and their use in therapy, including in pharmaceutical combinations, especially in the treatment of bacterial, e.g. pneumococcal, infections.

### BACKGROUND OF THE INVENTION

Streptococcus pneumoniae (pneumococcus) is one of the most potent human pathogens, affecting over 10 million people worldwide, of all age groups, in particular young children, the elderly and the immunocompromised. It is a leading causative agent of serious, often fatal diseases, such as pneumonia, bacteraemia and meningitis. It is also responsible of other less serious, but nevertheless debilitating diseases such as otitis media and keratitis.

Even after decades of using antibiotics and steroids as adjunctive to antibiotics the mortality and morbidity from pneumococcal diseases remains very high in the developed world and alarmingly high in the developing world. Nearly 20% of hospitalised patients still die despite antibiotic killing of the pneumococcus, while many survivors of pneumococcal meningitis suffer severe neurological handicaps, including cognitive impairment, vision and hearing loss, hence imposing huge distress on patients and their families and a very significant cost to healthcare systems. Today, infection with pneumococcus remains a major global public health problem that is widely recognised by leaders in the field and by health organisations, including the WHO.

One of the leading factors for this consistently high mortality and morbidity that is not addressed by the current standard therapy, is the toxaemia resulting from the release of toxic pneumococcal products, the most important of which is the pneumococcal toxin pneumolysin. This toxin is a major player in pneumococcal virulence and is the primary direct and indirect cause of toxaemia.

Pneumolysin belongs to the family of cholesterol dependent cytolysins (CDCs), which bind to cholesterol containing membranes and generate large pores that have lethal and sub-lethal effects on the affected cells. In the bacterium, the toxin pneumolysin is cytoplasmic and is mainly released from the pneumococcus after its lysis. Consequently, under the effect of lytic antibiotics, a large bolus of toxin is released, compounding the toxaemia. Thus, even if treatment with antibiotics is successful in clearing the bacteria from the patients, the subsequent release of the toxin is detrimental and can be fatal or cause long-term handicaps.

This toxaemia constitutes a substantial unmet medical need that is internationally recognised. Currently, corticosteroids, principally dexamethasone, are used as an adjunctive to antibiotic therapy for pneumococcal meningitis. However, even when dexamethasone is used, significant mortality and morbidity are seen and the widespread use of dexamathasone is still debated due to its non-specific effect, limited clinical impact and in some cases its detrimental effect in increasing neuronal apoptosis in meningitis [Lancet (2002) 360 211-218]. Therefore, the present state of the art is not adequate for the efficient treatment of invasive pneumococcal diseases.

There is considerable evidence substantiating the validity of pneumolysin as a therapeutic target. In the laboratories of the inventors it has been demonstrated that, using a mouse pneumonia model, a mutated strain of S. pneumoniae (PLN-A) that does not produce pneumolysin is no longer lethal, causes substantially less bacteraemia and exhibits a significant reduction in the severity of pulmonary inflammation. Other evidence obtained in a rat meningitis model, has shown that infection with the pneumolysin-negative mutant was markedly less severe than with wild-type pneumococci, with no observed damage to the ciliated epithelium of the brain and no apoptosis of the cells surrounding the epithelium [J. Infect, (2007) 55 394-399]. In pneumococcal meningitis in guinea pigs, wild-type pneumococci induced severe cochlear damage and hearing loss, while infection with PLN-A left the organ of Corti intact [Infect. Immun. (1997) 65 4411-4418]. An ex vivo model using cultured ciliated brain epithelial cells, enabled recreation of the in vivo situation, where cells lining the brain ventricles are exposed to S. pneumoniae. Both intact and antibiotic-killed wild-type pneumococci induced damage to the epithelial cells in culture and significantly impaired ciliary beating; effects not seen with PLN-A [Infect. Immun. (2000) 68 1557-1562]. This damaging effect of antibiotic-lysed pneumococci on the cultured ependymal cells is clearly caused by the toxin pneumolysin released from the antibiotic-lysed bacteria, as this damage was abolished in the presence of anti-pneumolysin antibodies [Infect. Immun. (2004) 72 6694-6698]. This finding supports the strategy that antibiotic-induced toxaemia is prevented by combination with anti-pneumolysin agents.

Evidence for the significant involvement of pneumolysin in pneumococcal infections and the substantial improvement of the disease prognosis in the absence of pneumolsyin, has led to the conclusion that pneumolysin constitutes a potential therapeutic target to develop new treatments for pneumococcal diseases. Previous research has shown the ability of cholesterol to inhibit pneumolysin [Biochem. J. (1974) 140 95-98], however, this inhibition is merely due to the fact that cholesterol is a natural cellular receptor of pneumolysin that is required for the pore formation in the target cell membrane. The topical application of cholesterol on the cornea of rabbits demonstrated a positive therapeutic effect in pneumococcal keratitis [Invest. Ophtalmol. Vis. Sci. (2007) 48 2661-2666]. This indicates the involvement of pneumolysin in pneumococcal keratitis and the therapeutic benefit obtained following its inhibition. However, cholesterol is not considered as a therapeutic agent for the treatment of pneumococcal diseases and has not been clinically used in patients. Another pneumolysin inhibitor, Allicin, a component in garlic extract, has been previously found to inhibit the haemolytic activity of pneumolysin in vitro [Toxicon (2011) 57 540-545]. This compound is a cysteine inhibitor that irreversibly binds to the reactive thiol group of the toxin. Compounds exhibiting such a property are unfavourable as drug candidates because of their potential unspecific binding to other cysteine-containing proteins in the body.

There remains a need to provide inhibitors of cytolysins, such as pneumolysin, which are suitable for use in the treatment of bacterial infections.

International Patent Application PCT/GB2012/053022, published after the priority date of the present application and herein incorporated by reference in its entirety, discloses N-phenyl substituted pyrrole derivatives as cytolysin inhibitors, that specifically inhibit the direct toxic effect of pneumolysin and other cholesterol dependent cytolysins that are pivotal in the virulence of their respective hosts, including the compound 2,5-bis(dimethylcarbamoyl)-1-(4-methoxyphenyl)-1H-pyrrole-3,4-diyl bis(4-((phosphonooxy)-methyl)benzoate). These compounds have no structural similarity to Allicin and do not bind covalently to the reactive thiol groups of the toxins.

The present invention provides novel N-phenyl substituted pyrrole cytolysin inhibitors which demonstrates particularly advantageous properties e.g. in terms of solubility and physicochemical properties making them particularly suitable for parenteral delivery. The compounds of the present invention also prevent stimulation of host-derived toxic effects induced by pneumolysin and, it may be assumed, other cholesterol dependent cytolysins. Thus the compounds may be used as single agents or as an adjunct to antibiotics, to prevent or attenuate pneumolysin-induced toxicity and its anti-host effects seen during infections caused e.g. by S. pneumoniae.

### SUMMARY OF THE INVENTION

According to the invention there is provided a compound of formula (I):

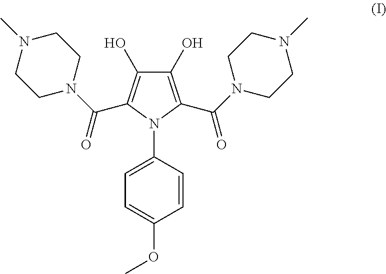

or a pharmaceutically acceptable prodrug derivative thereof, or a pharmaceutically acceptable salt or solvate thereof.

In a further aspect, the present invention provides a compound of formula (I) or a pharmaceutically acceptable prodrug derivative thereof, or a pharmaceutically acceptable salt or solvate thereof (hereinafter referred to as a compound of the invention) for use as a medicament.

### DETAILED DESCRIPTION OF THE INVENTION

Prodrug derivatives of compounds of the invention will break down after administration to a subject to form an active compound of formula (I) (sometimes referred to herein as “parent active compound”) in vivo. Prodrug derivatives of compounds of the invention may have some intrinsic biological activity (e.g. as pneumolysin inhibitors) however typically they have little or no such intrinsic activity.

Prodrug derivatives of the compounds of formula (I) include ester prodrug derivatives. Ester prodrug derivatives include carboxylate ester, sulfamate ester, phosphate ester and carbamate ester derivatives, preferably carboxylate ester, sulfamate ester or phosphate ester derivatives, more preferably carboxylate ester or phosphate ester derivatives, even more preferably carboxylate ester derivatives.

Examples of ester prodrug derivatives include compounds of formula (Ia):

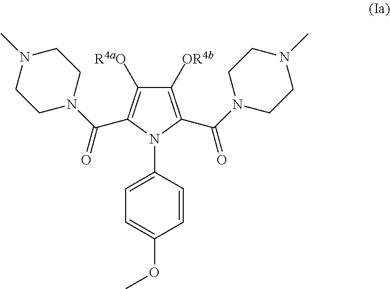

wherein one or both of R^{4a} and R^{4b} are independently selected from —C(O)R¹⁶, —SO₂NH₂, —PO(OR¹⁹)(OR²⁰), —CHR²⁶—OPO(OR¹⁹)(OR²⁰) where R²⁶ is hydrogen or C₁-C₆ alkyl, and —C(O)NR¹⁷R¹⁸, wherein R¹⁶, R¹⁷, R¹⁸, R¹⁹ and R²⁰ are independently selected from:
(a) C₁-C₆ alkyl, C₂-C₆ alkenyl, C₂-C₆ alkynyl, C₃—C₁₀ cycloalkyl, C₅-C₁₀ cycloalkenyl, heterocyclyl, —C₁-C₃ alkyl-C₃-C₁₀ cycloalkyl, —C₁-C₃ alkyl-C₅-C₁₀ cycloalkenyl or —C₁-C₃ alkylheterocyclyl, or R¹⁷ and R¹⁸ together with the N to which they are attached may form a 5- or 6-membered heterocyclic ring optionally containing a further heteroatom selected from O, S and NR^{25a}R^{25b} where R^{25a} is hydrogen, C₁-C₆ alkyl, —CH₂—OPO(OR¹⁹)(OR²⁰) or a 5- or 6-membered heterocyclic ring, and R^{25b} is absent or C₁-C₆ alkyl; and in which any of the aforementioned R¹⁶, R¹⁷ or R¹⁸ groups may be optionally substituted by one or more groups selected from cyano, —OPO(OR¹⁹)(OR²⁰), —(O(CH₂)_{z})ᵣOR²⁴, wherein each z, which may be the same or different, represents 2 or 3, r represents an integer selected from 1 to 20, and R²⁴ is hydrogen, C₁-C₃ alkyl or —PO(OR¹⁹)(OR²⁰), C₁-C₆ alkoxy, C₁-C₆ fluoroalkoxy, C₁-C₆ alkyl, C₁-C₆ fluoroalkyl and —C(O)NR^{a}R^{b}, where R^{a} and R^{b} are independently selected from hydrogen and C₁-C₆ alkyl, and any of the aforementioned R¹⁶, R¹⁷ R or R¹⁸ groups may be optionally substituted by one or more halogen atoms; and
(b) aryl, heteroaryl, C₁-C₃ alkylaryl and —C₁-C₃ alkylheteroaryl, said aryl and heteroaryl groups being optionally substituted;

or R¹⁸, R¹⁹ and R²⁰ may independently represent hydrogen;

and wherein when one of R^{4a} and R^{4b} are independently selected from the groups defined above the other is hydrogen.

Optional substituents for phenyl, aryl and heteroaryl groups within the definitions of R¹⁶, R¹⁷, R¹⁸, R¹⁹ and R²⁰ are suitably selected from hydroxyl, halo, cyano, —(CHR²⁶)_{q}—OPO(OR¹⁹)(OR²⁰) wherein q represents 0 or 1 (said group not being substituted by another R¹⁹ or R²⁰ containing group), C₁-C₆ alkoxy or C₁-C₆ fluoroalkoxy, e.g. C₁-C₃ alkoxy or C₁-C₃ fluoroalkoxy such as methoxy, ethoxy or trifluoromethoxy, C₁-C₆ alkyl or C₁-C₆ fluoroalkyl, e.g. C₁-C₃ alkyl or C₁-C₃ fluoroalkyl such as methyl or trifluoromethyl, and —C(O)NR^{a}R^{b}, where R^{a} and R^{b} are independently selected from hydrogen and C₁-C₆ alkyl e.g. C₁-C₃ alkyl such as methyl; and also when two adjacent hydroxyl substituents are present they may optionally be connected by a methylene group to form an acetal. Another possible optional substituent is —SF₅. Said aryl and heteroaryl groups, if substituted, may be substituted by 1, 2 or 3, preferably 1 or 2, more preferably 1 substituent.

Optional substituents for the C₁-C₆ alkyl, C₂-C₆ alkenyl, C₂-C₆ alkynyl, C₃-C₁₀ cycloalkyl, C₅-C₁₀ cycloalkenyl, heterocyclyl, —C₁-C₃ alkyl-C₃-C₁₀ cycloalkyl, —C₁-C₃ alkyl-C₅-C₁₀ cycloalkenyl, —C₁-C₃ alkylheterocyclyl or heterocyclic ring groups of R¹⁶, R¹⁷, R¹⁸, R¹⁹ and R²⁰ include substituents selected from cyano, —OPO(OR¹⁹)(OR²⁰) (said group not being substituted by another R¹⁹ or R²⁰ containing group), C₁-C₆ alkoxy or C₁-C₆ fluoroalkoxy, e.g. C₁-C₃ alkoxy or C₁-C₃ fluoroalkoxy such as methoxy, ethoxy or trifluoromethoxy, C₁-C₆ alkyl or C₁-C₆ fluoroalkyl, e.g. C₁-C₃ alkyl or C₁-C₃ fluoroalkyl such as methyl or trifluoromethyl, and —C(O)NR^{a}R^{b}, where R^{a} and R^{b} are independently selected from hydrogen and C₁-C₆ alkyl e.g. C₁-C₃ alkyl such as methyl. Optional substituents for the groups R⁵, R⁶ and R⁷ also include one or more (e.g. 1, 2, or 3) halogen atoms e.g. F or Cl atoms (especially F atoms).

R¹⁶ preferably represents C₁-C₆ alkyl or C₃-C₁₀ cycloalkyl in which either of the aforementioned groups may be optionally substituted (and is preferably substituted) by a group selected from —OPO(OR¹⁹)(OR²⁰) and —(O(CH₂)_{z})ᵣOR²⁴, where each z, which may be the same or different, represents 2 or 3, r represents an integer selected from 1 to 20, e.g. 7 to 12, and R²⁴ is hydrogen, C₁-C₃ alkyl or —PO(OR¹⁹)(OR²⁰).

Alternatively, R¹⁶ preferably represents phenyl optionally substituted (and is preferably substituted) by —(CHR²⁶)_{q}—OPO(OR¹⁹)(OR²⁰) wherein q represents 0 or 1.

R¹⁷ preferably represents C₁-C₆ alkyl e.g. methyl. R¹⁸ preferably represents C₁-C₆ alkyl e.g. methyl. Alternatively, R¹⁷ and R¹⁸ together with the N to which they are attached may form a 5- or 6-membered heterocyclic ring optionally containing a further heteroatom selected from O, S and NR^{25a} where R^{25a} is hydrogen, C₁-C₆ alkyl, —CH₂—OPO(OR¹⁹)(OR²⁰) or a 5- or 6-membered heterocyclic ring.

R¹⁹ is preferably hydrogen, methyl or ethyl, especially hydrogen.

R²⁰ is preferably hydrogen, methyl or ethyl, especially hydrogen.

R^{25a} is preferably hydrogen or methyl.

R^{25b} is preferably absent.

R²⁶ is preferably hydrogen or methyl, more preferably methyl.

In one embodiment q represents 0. In another embodiment q represents 1.

In one embodiment one of R^{4a} and R^{4b} represents a prodrug derivative group as defined above. In another embodiment both of R^{4a} and R^{4b} represent a prodrug group as defined above.

In one embodiment both of R^{4a} and R^{4b} are independently selected from —C(O)R¹⁶, —SO₂NH₂, —PO(OR¹⁹)(OR²⁰), —CHR²⁶—OPO(OR¹⁹)(OR²⁰) where R²⁶ is hydrogen or C₁-C₆ alkyl, and —C(O)NR¹⁷R¹⁸. In a further embodiment one of R^{4a} and R^{4b} is selected from —C(O)R¹⁶, —SO₂NH₂, —PO(OR¹⁹)(OR²⁰), —CHR²⁶—OPO(OR¹⁹)(OR²⁰) where R²⁶ is hydrogen or C₁-C₆ alkyl, and —C(O)NR¹⁷R¹⁸; and the other of R^{4a} and R^{4b} is hydrogen.

One or both of R^{4a} and R^{4b} are preferably independently selected from —C(O)R¹⁶.

When the prodrug is a carboxylate ester prodrug, e.g. wherein one or both of R^{4a} and R^{4b} are —C(O)R¹⁶, the carbon atom adjacent to the C(O) moiety is preferably a tertiary or quaternary carbon atom.

Specific examples of prodrug derivatives include compounds of formula (Ia) wherein one or both of R^{4a} and R^{4b} are independently selected from —SO₂NH₂, —PO(OH)₂, —CH₂—PO(OH)₂, —PO(OEt)₂, —CON-(4-N-piperidinyl-piperidine), —COt-butyl, —COisopropyl, —CON—(N-methyl)piperazine, —CON-piperazine, —CON(CH₃)₂, COCH₃, —CO—(CH₂)₂—OMe, —CO(CH₂)₂—(O(CH₂)₂)ₚOMe where p is 1 to 12, —CO—CMe₂—CH₂—(O(CH₂)₃)ₚOMe where p is 1 to 12, —CO—CMe₂—CH₂—(O(CH₂)₂)ₚO—PO(OH)₂ where p is 1 to 12, —CO—CMe₂—CH₂—(O(CH₂)₂)ₚO—PO(OH)₂ where p is 1 to 12, —CO-(4-phosphonoxymethylbenzene) and —CO-(4-phosphonoxymethylcyclohexane); wherein when only one of R^{4a} and R^{4b} represents a prodrug derivative group as defined above the other of R^{4a} and R^{4b} is hydrogen. A group of specific examples of prodrug derivatives include compounds of formula (Ia) wherein R^{4a} and R^{4b} are independently selected from —SO₂NH₂, —PO(OH)₂, —CON-(4-N-piperidinyl-piperidine), —COt-butyl, —COisopropyl, —CON—(N-methyl)piperazine, —CON(CH₃)₂ and COCH₃.

A particular prodrug of formula (Ia) which may be mentioned is 1-(4-methoxyphenyl)-2,5-bis(4-methylpiperazine-1-carbonyl)-1H-pyrrole-3,4-diylbis(2-methylpropanoate), i.e. the compound of formula (Ia) where R^{4a} and R^{4b} are —C(O)CH(CH₃)₂, or a salt or solvate thereof, for example the dihydrochloride salt thereof.

While the preferred groups for each variable have generally been listed above separately for each variable, preferred compounds of this invention include those in which several or each variable in formula (Ia) is selected from the preferred, more preferred or particularly listed groups for each variable. Therefore, this invention is intended to include all combinations of preferred, more preferred and particularly listed groups.

The molecular weight of the compounds of the invention is preferably less than 2000, more preferably less than 1000, even more preferably less than 800, for example less than 600.

Examples of salts of the compounds of the invention include all pharmaceutically acceptable salts prepared from pharmaceutically acceptable non-toxic bases or acids. Salts derived from bases include, for example, potassium and sodium salts and the like. Salts derived from acids, include those derived from inorganic and organic acids such as, for example, hydrochloric, methanesulfonic, sulfuric and p-toluenesulfonic acid and the like.

Examples of solvates of the compounds of the invention include hydrates.

The compound described herein includes one or more chiral centers, and the disclosure extends to include racemates, enantiomers and stereoisomers resulting therefrom. In one embodiment one enantiomeric form is present in a substantially purified form that is substantially free of the corresponding enantiomeric form.

The invention also extends to all polymorphic forms of the compounds of the invention.

The invention also extends to isotopically-labelled compounds of the invention in which one or more atoms are replaced by an atom having an atomic mass or mass number different from the atomic mass or mass number most commonly found in nature. Examples of isotopes that can be incorporated into compounds of the invention include isotopes of hydrogen, carbon, nitrogen, and phosphorus, such as ²H, ³H, ¹¹C, ¹⁴C, ¹⁵N, ³²P and ³³P. Isotopically labelled compounds of formula (I) may be prepared by carrying out the synthetic methods described below and substituting an isotopically labelled reagent or intermediate for a non-isotopically labelled reagent or intermediate.

The invention extends to all tautomeric forms of the compounds illustrated herein (particularly enol-keto tautomers).

The compounds of the invention may be prepared as described in the Examples and in the following general methods.

A compound of formula (I) may be prepared by reacting a compound of formula (II) or a protected derivative thereof:

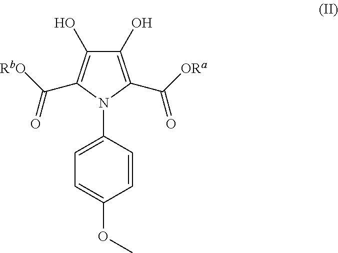

with 1-methylpiperazine, and if required deprotecting the resulting compound. The reaction may be conducted, for example, in the presence of isopropylmagnesium chloride and in a solvent such as THF.

A method for preparing compounds of formula (Ia) in which one or both of R^{4a} and R^{4b} represent groups other than hydrogen is shown below in Scheme A:

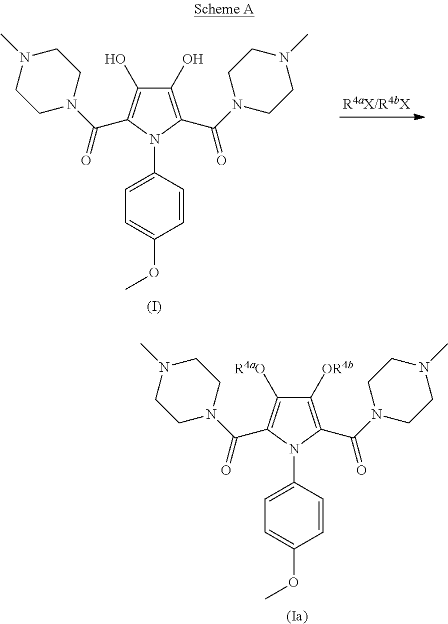

wherein X is a leaving group such as halogen, an ester (—OCOR′, giving a mixed anhydride), or hydrogen, when used in combination with a suitable coupling agent, such as: 1-ethyl-3-(3-dimethylaminopropyl)carbodiimide) (EDC), N,N′-diisopropylcarbodiimide (DIC) or 1,1′-carbonyldiimidazole (CDI). Suitably X is halogen.

Scheme A may be adapted to convert one or both hydroxyl groups to OR^{4a} and/or OR^{4b} depending on the molar excess of reagent(s) employed. When R^{4a} and R^{4b} are different, it may be necessary to employ a protection strategy to incorporate one and then the other group.

Thus according to a further aspect of the invention there is provided a process for the production of the compound of formula (Ia) which comprises reacting a compound of formula (I):

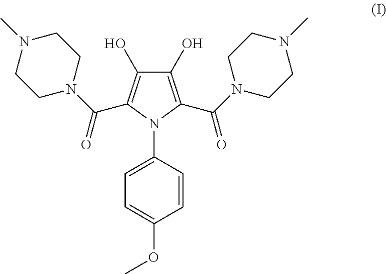

or a protected derivative thereof, with a compound of formula R^{4a}X and/or a compound of formula R^{4b}X, where X is independently a leaving group such as one mentioned above.

Any novel intermediates, such as those defined above, may be of use in the synthesis of compounds of the invention and are therefore also included within the scope of the invention.

Thus according to a further aspect of the invention there is provided a protected derivative of the compound of formula (I), e.g. the compound (3,4-bis(benzyloxy)-1-(4-methoxyphenyl)-1H-pyrrole-2,5-diyl)bis((4-methylpiperazin-1-yl)methanone).

Protecting groups may be required to protect chemically sensitive groups during the synthesis of the compound of the invention, to ensure that the process is efficient. Thus if desired or necessary, intermediate compounds may be protected by the use of conventional protecting groups. Protecting groups and means for their removal are described in “Protective Groups in Organic Synthesis”, by Theodora W. Greene and Peter G. M. Wuts, published by John Wiley & Sons Inc; 4^{th} Rev Ed., 2006, ISBN-10: 0471697540.

As indicated above the compounds of the invention are useful for treatment of bacterial infections caused by bacteria producing pore-forming toxins, such as cholesterol dependent cytolysins.

In particular the compounds of the invention are useful for the treatment of toxaemia associated with bacterial infections.

For such use the compounds of the invention will generally be administered in the form of a pharmaceutical composition.

Further, the present invention provides a pharmaceutical composition comprising a compound of the invention optionally in combination with one or more pharmaceutically acceptable diluents or carriers.

Diluents and carriers may include those suitable for parenteral, oral, topical, mucosal and rectal administration.

As mentioned above, such compositions may be prepared e.g. for parenteral, subcutaneous, intramuscular, intravenous, intra-articular or peri-articular administration, particularly in the form of liquid solutions or suspensions; for oral administration, particularly in the form of tablets or capsules; for topical e.g. intravitreal, pulmonary or intranasal administration, particularly in the form of eye drops, powders, nasal drops or aerosols and transdermal administration; for mucosal administration e.g. to buccal, sublingual or vaginal mucosa, and for rectal administration e.g. in the form of a suppository.

The compositions may conveniently be administered in unit dosage form and may be prepared by any of the methods well-known in the pharmaceutical art, for example as described in Remington's Pharmaceutical Sciences, 17th ed., Mack Publishing Company, Easton, Pa., (1985). Formulations for parenteral administration may contain as excipients sterile water or saline, alkylene glycols such as propylene glycol, polyalkylene glycols such as polyethylene glycol, oils of vegetable origin, hydrogenated naphthalenes and the like. Formulations for parenteral administration may be provided in solid form, such as a lyophilised composition, the lyophilised composition may be re-constituted, preferably just before administration. Re-constitution may involve dissolving the lyophilised composition in water or some other pharmaceutically acceptable solvent, for example physiological saline, an aqueous solution of a pharmaceutically acceptable alcohol, e.g. ethanol, propylene glycol, a polyethylene glycol, e.g. polyethylene glycol 300, and the like, or some other sterile injectable.

Formulations for nasal administration may be solid and may contain excipients, for example, lactose or dextran, or may be aqueous or oily solutions for use in the form of nasal drops or metered spray. For buccal administration typical excipients include sugars, calcium stearate, magnesium stearate, pregelatinated starch, and the like.

Compositions suitable for oral administration may comprise one or more physiologically compatible carriers and/or excipients and may be in solid or liquid form. Tablets and capsules may be prepared with binding agents, for example, syrup, acacia, gelatin, sorbitol, tragacanth, or poly-vinylpyrollidone; fillers, such as lactose, sucrose, corn starch, calcium phosphate, sorbitol, or glycine; lubricants, such as magnesium stearate, talc, polyethylene glycol, or silica; and surfactants, such as sodium lauryl sulfate. Liquid compositions may contain conventional additives such as suspending agents, for example sorbitol syrup, methyl cellulose, sugar syrup, gelatin, carboxymethyl-cellulose, or edible fats; emulsifying agents such as lecithin, or acacia; vegetable oils such as almond oil, coconut oil, cod liver oil, or peanut oil; preservatives such as butylated hydroxyanisole (BHA) and butylated hydroxytoluene (BHT). Liquid compositions may be encapsulated in, for example, gelatin to provide a unit dosage form.

Solid oral dosage forms include tablets, two-piece hard shell capsules and soft elastic gelatin (SEG) capsules.

A dry shell formulation typically comprises of about 40% to 60% concentration of gelatin, about a 20% to 30% concentration of plasticizer (such as glycerin, sorbitol or propylene glycol) and about a 30% to 40% concentration of water. Other materials such as preservatives, dyes, opacifiers and flavours also may be present. The liquid fill material comprises a solid drug that has been dissolved, solubilized or dispersed (with suspending agents such as beeswax, hydrogenated castor oil or polyethylene glycol 4000) or a liquid drug in vehicles or combinations of vehicles such as mineral oil, vegetable oils, triglycerides, glycols, polyols and surface-active agents.

Pharmaceutical compositions of the invention may optionally include one or more anti-oxidants (e.g. ascorbic acid or metabisulfate and salts thereof).

Particular pharmaceutical compositions according to the invention which may be mentioned include the following:
A pharmaceutical composition for parenteral, e.g. intravenous, administration.
A pharmaceutical composition for oral administration.
A pharmaceutical composition for parenteral, e.g. intravenous, or oral administration in unit dose form.
A pharmaceutical composition for parenteral, e.g. intravenous, administration in solid form for reconstitution with a liquid prior to administration.
A pharmaceutical composition for parenteral, e.g. intravenous, administration in liquid form e.g. a solution.

The compounds of the invention are inhibitors of the cholesterol-dependent cytolysin, pneumolysin, produced by the bacterium Streptococcus pneumoniae. It also inhibits Streptolysin O (SLO) produced by Group A Streptococci and Perfringolysin O (PFO) produced by Clostridium perfringens. It is also expected to inhibit other members of the closely related cholesterol-dependent cytolysins, examples of which include, but are not limited to, Listeriolysin O (LLO) produced by Listeria monocytogenes, Anthrolysin O (ALO) produced by Bacillus anthracis and Suilysin (SLY) produced by Streptococcus suis.

The compounds of the invention are useful for the treatment of bacterial infections, e.g. pneumococcal infections including the associated toxaemia where the pneumolysin toxin has been demonstrated to play a pivotal role in the diseases produced. Such diseases include, but are not limited to, pneumococcal pneumonia, pneumococcal meningitis, pneumococcal septicaemia/bacteraemia, pneumococcal keratitis and pneumococcal otitis media. The compound of the invention is also useful for the treatment of pneumococcal infections associated with other conditions. Such conditions include (without limitation) cystic fibrosis and chronic obstructive pulmonary disease (COPD). For example, S. pneumoniae has been isolated from patients with COPD and is believed to be an exacerbatory factor in this disease.

The compounds of the invention are useful for the treatment of infections caused by group A Streptococci (GAS), including but not limited to, invasive group A Streptococcal diseases, where the toxin Streptolysin O (SLO) has been demonstrated to play a crucial role in the pathogenesis of systemic GAS diseases.

The compounds of the invention are useful for the treatment of infections caused by Clostridium perfringens including, but not limited to, gas gangrene, characterized by myonecrosis, septic shock and death, where the toxin Perfringolysin O has been demonstrated to be a major virulence factor in the pathogenesis of this disease.

The compounds of the invention are useful for the treatment of infections caused by Bacillus anthracis, where the cholesterol dependent cytolysin Anthrolysin O (ALO) plays an essential role in gastrointestinal (GI) anthrax, and contributes to the pathogenesis of inhalational anthrax.

The compounds of the invention are useful for the treatment of other diseases caused by Gram positive bacteria, producing cholesterol-dependent cytolysins, examples of which include, but are not limited to:

Porcine meningitis, septicaemia/bacteraemia and septic shock caused by Streptococcus suis which produces a cholesterol dependent cytolysin, Suilysin, involved in the pathogenesis of diseases by S. suis.

Encephalitis, enteritis, meningitis, septicaemia/bacteraemia and pneumonia caused by Listeria monocytogenes where the cholesterol dependent cytolysin, listeriolosin O (LLO), plays an important role in the pathogensis of the above diseases.

The compounds of the invention may well also be useful for the inhibition of other bacterial pore-forming toxins, such as the RTX family of toxins, which are essential in the virulence of their host. Examples include, but are not limited to, pneumonia and septicaemia/bacteraemia caused by Staphylococcus aureus, which produces the pore-forming toxin staphylococcal c-hemolysis and peritonitis caused by pathogenic Escherichia coli which produces the pore forming toxin α-hemolysin.

Thus the invention provides:
The compounds of the invention for use in the treatment of bacterial infections caused by bacteria producing pore-forming toxins, wherein the bacterial infection is caused by Streptococcus spp. (e.g. Streptococcus pneumoniae, Group A Streptococci or Streptococcus suis), Clostridium spp. (e.g. Clostridium perfringens), Listeria spp. (e.g. Listeria monocytogenes) or Bacillus spp. (e.g. Bacillus anthracis);
The compounds of the invention for the treatment of bacterial infection which is caused by Streptococcus pneumonia;
The compounds of the invention for use in the treatment of pneumococcal pneumonia, pneumococcal meningitis, pneumococcal septicaemia/bacteraemia, pneumococcal keratitis or pneumococcal otitis media; and
The compounds of the invention for the treatment of conditions selected from gas gangrene, gastrointestinal anthrax, inhalational anthrax, porcine meningitis, encephalitis, septicaemia/bacteraemia and pneumonia which are caused by bacteria other than pneumococcus.

The compounds of the invention may be used to treat either humans or animals, such as domestic animals or livestock, e.g. pigs, cows, sheep, horses etc, and references to pharmaceutical compositions should be interpreted to cover compositions suitable for either human or animal use.

Thus, in a further aspect, the present invention provides a compound of the invention for use in the treatment of the above mentioned conditions.

In a further aspect, the present invention provides a compound of the invention for the manufacture of a medicament for the treatment of the above mentioned conditions.

In a further aspect, the present invention provides a method of treatment of the above mentioned conditions which comprises administering to a subject in need thereof an effective amount of a compound of the invention or a pharmaceutical composition thereof.

The word “treatment” is intended to embrace prophylaxis as well as therapeutic treatment.

The compounds of the invention may be used either alone or in combination with further therapeutically active ingredients. Thus compounds of the invention may be administered in combination, simultaneously, sequentially or separately, with further therapeutically active ingredients either together in the same formulation or in separate formulations and either via the same route or via a different route of administration. The compounds of the invention may thus be administered in combination with one or more other active ingredients suitable for treating the above mentioned conditions. For example, possible combinations for treatment include combinations with antimicrobial agents, e.g. antibiotic agents, including natural, synthetic and semisynthetic antimicrobial agents. Examples of antibiotic agents include β-lactams including, but not limited to, penicillin, benzylpenicillin, amoxicillin and all generations thereof; β-lactams in combination with β-lactamase inhibitors including, but not limited to, clavulanic acid and sulbactam; cephalosporins including, but not limited to, cefuroxime, cefotaxime and ceftriaxone; fluoroquinolones including, but not limited to, levofloxacin and moxifloxacin; tetracyclines including, but not limited to, doxycycline; macrolides including, but not limited to, erythromycin and clarithromycin; lipopeptide antibiotics including, but not limited to, daptomycin; aminoglycosides including, but not limited to, kanamycin and gentamicin; glycopeptide antibiotics, including but not limited to, vancomycin; lincosamides including, but not limited to, clindamycin and lincomycin; rifamycins including, but not limited to, rifampicin; and chloramphenicol.

Further combinations include combinations with immunomodulatory agents, such as anti-inflammatory agents.

Immunomodulatory agents can include for example, agents which act on the immune system, directly or indirectly, by stimulating or suppressing a cellular activity of a cell in the immune system, for example, T-cells, B-cells, macrophages, or antigen presenting cells, or by acting upon components outside the immune system which, in turn, stimulate, suppress, or modulate the immune system, for example, hormones, receptor agonists or antagonists and neurotransmitters, other immunomodulatory agents can include immunosuppressants or immunostimulants. Anti-inflammatory agents include, for example, agents which treat inflammatory responses, tissue reaction to injury, agents which treat the immune, vascular or lymphatic systems or combinations thereof. Examples of anti-inflammatory and immunomodulatory agents include, but are not limited to, interferon derivatives such as betaseron, β-interferon, prostane derivatives such as iloprost and cicaprost, corticosteroids such as prednisolone, methylprednisolone, dexamethasone and fluticasone, COX2 inhibitors, immunsuppressive agents such as cyclosporine A, FK-506, methoxsalene, thalidomide, sulfasalazine, azathioprine and methotrexate, lipoxygenase inhibitors, leukotriene antagonists, peptide derivatives such as ACTH and analogs, soluble TNF (tumor necrosis factor) -receptors, TNF-antibodies, soluble receptors of interleukines, other cytokines and T-cell-proteins, antibodies against receptors of interleukins, other cytokines and T-cell-proteins. Further anti-inflammatory agents include non-steroidal anti-inflammatory drugs (NSAID's). Examples of NSAID's include sodium cromoglycate, nedocromil sodium, phosphodiesterase (PDE) inhibitors e.g. theophylline, PDE4 inhibitors or mixed PDE3/PDE4 inhibitors, leukotriene antagonists, inhibitors of leukotriene synthesis such as montelukast, iNOS inhibitors, tryptase and elastase inhibitors, beta-2 integrin antagonists and adenosine receptor agonists or antagonists such as adenosine 2a agonists, cytokine antagonists e.g. chemokine antagonists, such as CCR3 antagonists, or inhibitors of cytokine synthesis, and 5-lipoxygenase inhibitors.

Thus an aspect of the invention provides a compound of the invention in combination with one or more further active ingredients, for example one or more of the active ingredients described above.

Another aspect of the invention provides a pharmaceutical composition comprising a compound of the invention optionally in combination with one or more pharmaceutically acceptable adjuvants, diluents or carriers and comprising one or more other therapeutically active ingredients.

Similarly, another aspect of the invention provides a combination product comprising:

(A) a compound of the invention; and

(B) another therapeutic agent,

wherein each of components (A) and (B) is formulated in admixture with a pharmaceutically-acceptable adjuvant, diluent or carrier.

In this aspect of the invention, the combination product may be either a single (combination) pharmaceutical formulation or a kit-of-parts.

Thus, this aspect of the invention encompasses a pharmaceutical formulation including a compound of the invention and another therapeutic agent, in admixture with a pharmaceutically acceptable adjuvant, diluent or carrier (which formulation is hereinafter referred to as a “combined preparation”).

It also encompasses a kit of parts comprising components:
(i) a pharmaceutical formulation including a compound of the invention in admixture with a pharmaceutically acceptable adjuvant, diluent or carrier; and
(ii) a pharmaceutical formulation including another therapeutic agent, in admixture with a pharmaceutically-acceptable adjuvant, diluent or carrier;

which components (i) and (ii) are each provided in a form that is suitable for administration in conjunction with the other.

Component (i) of the kit of parts is thus component (A) above in admixture with a pharmaceutically acceptable adjuvant, diluent or carrier. Similarly, component (ii) is component (B) above in admixture with a pharmaceutically acceptable adjuvant, diluent or carrier.

The other therapeutic agent (i.e. component (B) above) may be, for example, any of the agents e.g. antimicrobial or immunomodulatory agents mentioned above.

The combination product (either a combined preparation or kit-of-parts) of this aspect of the invention may be used in the treatment or prevention of any of the conditions mentioned above.

The compound of the invention may also be provided for use, e.g. with instructions for use, in combination with one or more further active ingredients.

Thus a further aspect of the invention provides a compound of the invention for use in combination with one or more further active ingredients, for example one or more of the active ingredients described above.

The compound of the invention for use in this aspect of the invention may be used in the treatment or prevention of any of the conditions mentioned above.

The invention will now be described by reference to the following examples which are for illustrative purposes and are not to be construed as a limitation of the scope of the present invention.

### EXAMPLES

Abbreviations

AcOH glacial acetic acid

aq. aqueous

Bn benzyl

br broad

Boc tert-butoxycarbonyl

COPD chronic obstructive pulmonary disease

d doublet

DCM dichloromethane

DIPEA N,N-diisopropylethylamine

DMAP 4-dimethylaminopyridine

DMF N,N-dimethylformamide

DMSO dimethylsulfoxide

EDC 1-ethyl-3-(3-dimethylaminopropyl)carbodiimide

EtOAc ethyl acetate

h hour(s)

HATU N,N,N′,N′-tetramethyl-O-(7-azabenzotriazol-1-yl)uronium PF₆

HPLC high performance liquid chromatography

m multiplet

MeCN acetonitrile

MeOH methanol

min minute(s)

NMR nuclear magnetic resonance

PBS phosphate buffered saline

quin. quintet

RT room temperature

s singlet

sat. saturated

SAX solid supported strong cation exchange resin

sept. septet

sext. sextet

t triplet

TFAA trifluoroacetic acid anhydride

THF tetrahydrofuran

UV ultra violet

General Procedures

All starting materials and solvents were obtained from commercial sources or prepared according to literature conditions.

Hydrogenations were performed either on a Thales H-cube flow reactor or with a suspension of the catalyst under a balloon of hydrogen.

Column chromatography was performed on pre-packed silica (230-400 mesh, 40-63 μM) cartridges.

PBS solutions for solubility and stability studies were prepared by dissolving 1 Oxoid™ tablet (obtained from Thermo Scientific) in deionised water (100 mL).

Stability studies were carried out by dissolving 1-2 mg of compound in DMSO (1 mL) followed by addition of 0.4 mL of the resulting solution to stirred PBS solution (9.6 mL) at 37.5° C. A sample (ca. 0.5 mL) was immediately taken for HPLC analysis. Further samples were then taken for analysis at various timepoints thereafter. Half-lives were determined from the decrease in concentration of compound with respect to time.

Analytical Methods

Analytical HPLC was carried out using an Agilent Zorbax Extend C18, Rapid Resolution HT 1.8 μm column eluting with a 5-95% gradient of either 0.1% formic acid in MeCN in 0.1% aqueous formic acid or a 5-95% gradient of MeCN in 50 mM aqueous ammonium acetate. Alternatively, a Waters Xselect CSH C18 3.5 μm eluting with a 5-95% gradient of 0.1% formic acid in MeCN in 0.1% aqueous formic acid. UV spectra of the eluted peaks were measured using either a diode array or variable wavelength detector on an Agilent 1100 system.

Analytical LCMS was carried out using an Agilent Zorbax Extend C18, Rapid Resolution HT 1.8 μm column eluting with a 5-95% gradient of either 0.1% formic acid in MeCN in 0.1% aqueous formic acid or a 5-95% gradient of MeCN in 50 mM aqueous ammonium acetate. Alternatively, a Waters Xselect CSH C18 3.5 μm eluting with a 5-95% gradient of 0.1% formic acid in MeCN in 0.1% aqueous formic acid. UV and mass spectra of the eluted peaks were measured using a variable wavelength detector on either an Agilent 1100 with or an Agilent Infinity 1260 LC with 6120 quadrupole mass spectrometer with positive and negative ion electrospray.

¹H NMR Spectroscopy:

NMR spectra were recorded using a Bruker Avance III 400 MHz instrument, using either residual non-deuterated solvent or tetra-methylsilane as reference.

Chemical Synthesis:

Compounds of the invention were prepared using the following general methods:

### EXAMPLE A1

### (3,4-Dihydroxy-1-(4-methoxyphenyl)-1H-pyrrole-2,5-diyl)bis((4-methylpiperazin-1-yl)methanone) (UL7-001)

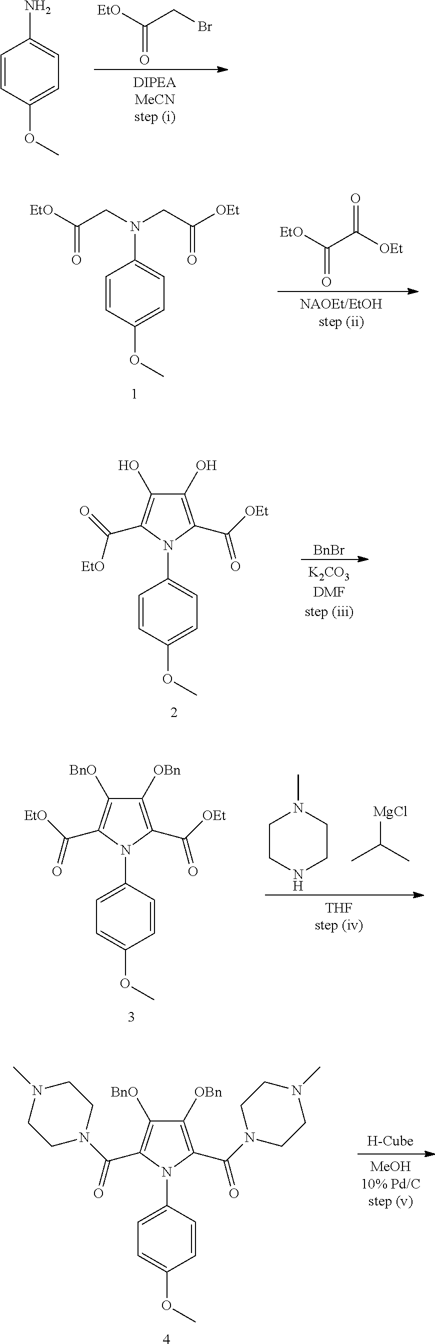

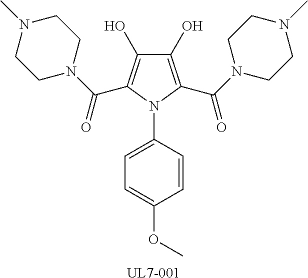

### Step (i): Diethyl 2,2′((4-methoxyphenyl)azanediyl)diacetate (1)

Ethyl 2-bromoacetate (146 mL, 1.30 mol) was added dropwise to a stirred solution of 4-methoxyaniline (75.0 g, 0.610 mol) and DIPEA (265 mL, 1.50 mol) in MeCN (300 mL). The reaction mixture was stirred at 60° C. for 16 h and then partitioned between 2M HCl_{(aq.)} (500 mL), and EtOAc (300 mL), the aqueous phase was extracted with EtOAc (300 mL) and the combined organics were washed succesively with 2M HCl_{(aq.)} (2×300 mL), water (500 mL), and brine (500 mL), dried (MgSO₄), filtered and solvents removed in vacuo to give diethyl 2,2′-((4-methoxyphenyl)azanediyl)diacetate (1) (180 g, 100%) as a purple oil: m/z 296 (M+H)⁺ (ES⁺). ¹H NMR (400 MHz, CDCl₃) δ: 6.82-6.78 (m, 2H), 6.64-6.59 (m, 2H), 4.19 (q, J=7.1 Hz, 4H), 4.10 (s, 4H), 3.74 (s, 3H), 1.27 (t, J=7.1 Hz, 6H).

### Step (ii): Diethyl 3,4-dihydroxy-1-(4-methoxyphenyl)-1H-pyrrole-2,5-dicarboxylate (2)

Diethyl oxalate (83.0 mL, 0.610 mol) was added dropwise to a stirred solution of diethyl 2,2′-((4-methoxyphenyl)azanediyl)diacetate (1) (180 g, 0.610 mol) in NaOEt (21% by wt in EtOH) (506 mL, 1.30 mol), the mixture was stirred at 100° C. for 1 h. The reaction was quenched with acetic acid (210 mL, 3.70 mol) and the resulting suspension was poured into iced water (1 L), the resulting off-white solid collected by vacuum filtration. The crude product was recrystallised from hot EtOH (3.50 L) to give diethyl 3,4-dihydroxy-1-(4-methoxyphenyl)-1H-pyrrole-2,5-dicarboxylate (2) (152 g, 71%) as a white solid: m/z 350 (M+H)⁺ (ES⁺). 348 (M−H)⁻ (ES). ¹H NMR (400 MHz, DMSO-d₆) δ: 8.64 (s, 2H), 7.13-7.01 (m, 2H), 6.92-6.81 (m, 2H), 3.99 (q, J=7.1 Hz, 4H), 3.78 (s, 3H), 0.99 (t, J=7.1 Hz, 6H).

### Step (iii): Diethyl 3,4-bis(benzyloxy)-1-(4-methoxyphenyl)-1H-pyrrole-2,5-dicarboxylate (3)

Benzyl bromide (42.6 mL, 358 mmol) was added dropwise to a stirred suspension of 3,4-dihydroxy-1-(4-methoxyphenyl)-1H-pyrrole-2,5-dicarboxylate (2) (50.0 g, 143 mmol) and K₂CO₃ (49.5 g, 358 mmol) in DMF (1 L), the reaction mixture was stirred at 60° C. for 4 h. After cooling to RT the reaction mixture was poured into ether (500 mL) and washed with brine (3×250 mL), dried (MgSO₄), filtered and concentrated in vacuo to afford a bright yellow solid. The crude product was triturated with isohexane to give diethyl 3,4-bis(benzyloxy)-1-(4-methoxyphenyl)-1H-pyrrole-2,5-dicarboxylate (3) (64.8 g, 85%) as a white solid: m/z 530 (M+H)⁺ (ES⁺). ¹H NMR (400 MHz, DMSO-d₆) δ: 7.48-7.29 (m, 10H), 7.17-7.09 (m, 2H), 6.95-6.87 (m, 2H), 5.09 (s, 4H), 3.99 (q, J=7.1 Hz, 4H), 3.80 (s, 3H), 0.99 (t, J=7.1 Hz, 6H).

### Step (iv): (3,4-Bis(benzyloxy)-1-(4-methoxyphenyl)-1H-pyrrole-2,5-diyl)bis((4-methylpiperazin-1-yl)methanone) (4)

To a stirred solution of diethyl 3,4-bis(benzyloxy)-1-(4-methoxyphenyl)-1H-pyrrole-2,5-dicarboxylate (3) (2.36 g, 4.46 mmol) and 1-methylpiperazine (2.47 mL, 22.3 mmol) in THF (50 mL) at 0° C. was added isopropylmagnesium chloride (11.1 mL, 22.3 mmol). The reaction mixture was allowed to warm to RT and stirred for 2 h. The reaction mixture was quenched with NH₄Cl_{(aq.)} (10 mL) and washed with brine (50 mL) followed by NaHCO_{3(aq.)} (50 mL). The organic layer was dried (MgSO₄), filtered and concentrated in vacuo. The residue was triturated with diethyl ether (50 mL) and the resultant solid was filtered, rinsing with diethyl ether, to afford (3,4-bis(benzyloxy)-1-(4-methoxyphenyl)-1H-pyrrole-2,5-diyl)bis((4-methylpiperazin-1-yl)methanone) (4) (2.02 g, 69%) as an off white solid: m/z 638 (M+H)⁺ (ES⁺). ¹H NMR (400 MHz, DMSO-d₆) δ: 7.41-7.30 (m, 10H), 7.03-6.98 (m, 2H), 6.97-6.92 (m, 2H), 5.00 (s, 4H), 3.76 (s, 3H), 3.42-3.26 (br m, 4H), 3.20-3.06 (br m, 4H), 2.16-1.84 (br m, 14H).

### Step (v): (3,4-Dihydroxy-1-(4-methoxyphenyl)-1H-pyrrole-2,5-diyl)bis((4-methylpiperazin-1-yl)methanone) (UL7-001)

A solution of (3,4-bis(benzyloxy)-1-(4-methoxyphenyl)-1H-pyrrole-2,5-diyl)bis((4-methylpiperazin-1-yl)methanone) (4) (2.01 g, 3.16 mmol) in methanol (50 mL) was hydrogenated in the H-Cube (10% Pd/C, 55×4 mm, Full hydrogen, 40° C., 1 mL/min) and concentrated in vacuo to afford (3,4-dihydroxy-1-(4-methoxyphenyl)-1H-pyrrole-2,5-diyl)bis((4-methylpiperazin-1-yl)methanone) (UL7-001) (1.42 g, 96%) as a yellow solid: m/z 458 (M+H)⁺ (ES⁺). ¹H NMR (400 MHz, DMSO-d₆) δ: 8.44 (s, 2H), 6.96-6.84 (m, 4H), 3.74 (s, 3H), 3.46-3.28 (br m, 8H), 2.23-2.07 (br m, 14H).

### EXAMPLE A2

### Alternative potential synthesis of (3,4-Dihydroxy-1-(4-methoxyphenyl)-1H-pyrrole-2,5-diyl)bis((4-methylpiperazin-1-yl)methanone) (UL7-001)

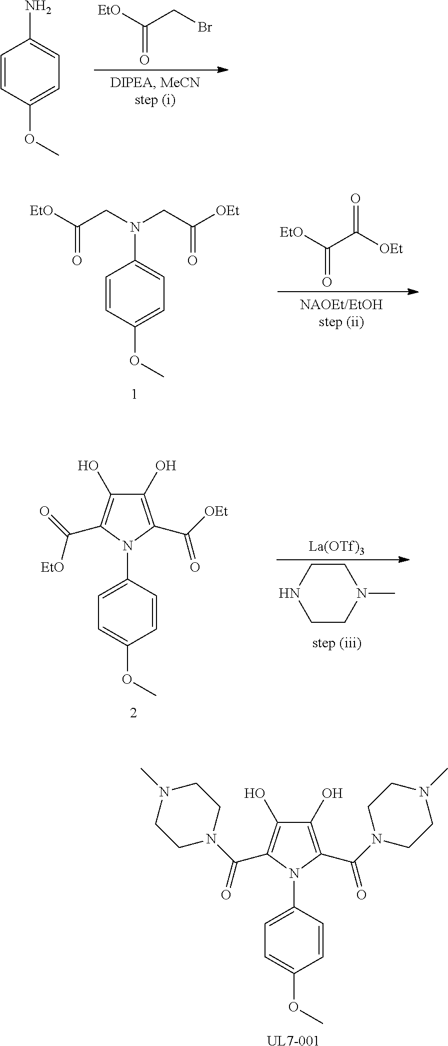

### EXAMPLE B

### 1-(4-Methoxyphenyl)-2,5-bis(4-methylpiperazine-1-carbonyl)-1H-pyrrole-3,4-diyl bis(2-methylpropanoate)dihydrochloride (UL7-002)

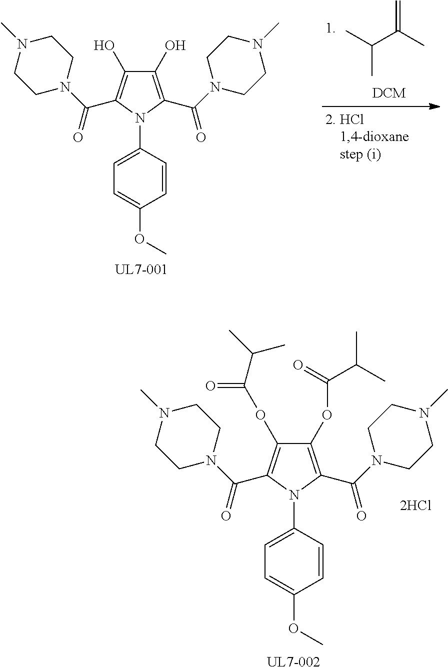

### Step (i): 1-(4-Methoxyphenyl)-2,5-bis(4-methylpiperazine-1-carbonyl)-1H-pyrrole-3,4-diylbis(2-methylpropanoate)dihydrochloride (UL7-002)

To a solution/suspension of (3,4-dihydroxy-1-(4-methoxyphenyl)-1H-pyrrole-2,5-diyl)bis((4-methylpiperazin-1-yl)methanone) (UL7-001) (1.25 g, 2.73 mmol) and 2-tert-Butylimino-2-diethylamino-1,3-dimethylperhydro-1,3,2-diazaphosphorine (polymer-bound,2.2 mmol/g) (3.73 g, 8.20 mmol) in DCM (20 mL) at 0° C. was added isobutyryl chloride (0.716 mL, 6.83 mmol). The mixture was allowed to warm to RT and shaken for 30 mins, after which it was filtered, washing with DCM, and the filtrate was concentrated in vacuo. The residue was dissolved in DCM (4 mL) and hydrogen chloride (1.37 mL, 5.46 mmol) (4M in 1,4-dioxane) was added dropwise. The mixture was stirred for 20 minutes, then concentrated in vacuo. The residue was triturated with diethyl ether (10 mL). The resultant solid was filtered, rinsing with diethyl ether, and dried in vacuo to afford1-(4-methoxyphenyl)-2,5-bis(4-methylpiperazine-1-carbonyl)-1H-pyrrole-3,4-diyl bis(2-methylpropanoate) dihydrochloride (UL7-002) (0.408 g, 22%) as an off-white solid: m/z 598 (M+H)⁺ (ES⁺). ¹H NMR (400 MHz, D₂O) δ: 7.39-3.30 (br m, 2H), 7.27-7.18 (br m, 2H), 4.60-4.36 (br m, 2H), 4.03-3.78 (br m, 5H), 3.68-3.29 (br m, 6H), 3.22-2.60 (br m, 14H), 1.28 (d, J=7.0 Hz, 12H).

The following compounds in Table 1 were prepared using the methods provided above:

TABLE 1

| | | | IC₅₀ | | |
|---|---|---|---|---|---|
| Entry | Structure (preparation method) | Compound name | (μM) | ¹H NMR Data | Ionisation |
| 1 | 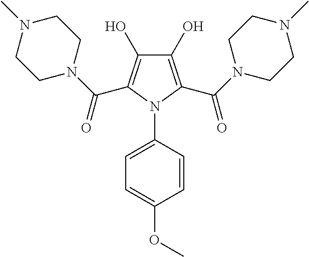 UL7-001 (Example A) | (3,4-Dihydroxy-1-(4- methoxyphenyl)-1H- pyrrole-2,5-diyl)bis((4- methylpiperazin-1- yl)methanone) | 0.3 | (DMSO-d₆) δ: 8.44 (s, 2H), 6.96-6.84 (m, 4H), 3.74 (s, 3H), 3.46-3.28 (br m, 8H), 2.23-2.07 (br m, 14H) | m/z 458 (M + H)⁺ (ES⁺) |
| 2 | 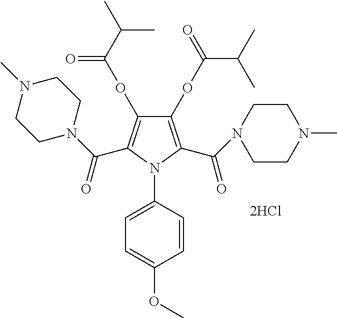 UL7-002 (Example B) | 1-(4-Methoxyphenyl)-2,5- bis(4-methylpiperazine- 1-carbonyl)-1H-pyrrole- 3,4-diyl bis(2- methylpropanoate) dihydrochloride | 6.8 | δ: 7.39-3.30 (brm, 2H), 7.27-7.18 (br m, 2H), 4.60- 4.36 (br m, 2H), 4.03-3.78 (br m, 5H), 3.68-3.29 (br m, 6H), 3.22-2.60 (br m, 14H), 1.28 (d, J = 7.0 Hz, 12H) | m/z 598 (M + H)⁺ (ES⁺) |

Biological Testing

There is provided below a summary of the biological assays performed with compounds of the invention.

A. Primary In Vitro Assay: Inhibition of the Haemolytic Activity of Pneumolysin

Rationale

The basis of this assay is that when pneumolysin is added to red blood cells, it induces their lysis and leads to the release of haemoglobin. In the presence of an inhibitory compound, pneumolysin-induced lysis is abolished, the red blood cells pellet at the bottom of the microtitre plate well and the supernatant is clear. However, if the compound is not inhibitory, the red blood cells are lysed and haemoglobin is released into the supernatant.

Experimental Procedure

Test compound solutions (typically at 5 mM in DMSO) were diluted 1:1 in 100% DMSO. The compounds were then two-fold serially diluted in 100% DMSO across 11 wells of 96-well round-bottomed microtitre plate. PBS was then added to all the wells to achieve a 1:10 dilution of the compound in PBS. Pneumolysin was then added at a concentration equal to its LD100. Plates were then incubated at 37° C. for 30-40 min. After the incubation period, an equal volume of 4% (v/v) sheep erythrocyte suspension was added to each well and the plates incubated again at 37° C., for at least 30 min. Controls with only erythrocytes in PBS (control for no lysis) or erythrocytes plus pneumolysin (control for lysis) were prepared following the same procedure. Following the incubation with the erythrocytes, the Absorbance at 595 nm of each well was measured and the data used to determine the IC₅₀ for each test compound. The IC₅₀ values were determined using non-linear regression curve fitting. For that, the Log of the concentrations of the test compound was plotted against the percentage inhibition, estimated from the A₅₉₅ values, followed by fitting a Hill Slope to the data.

Results

This assay is principally relevant for the determination of the inhibitory activity of the parent active compound UL7-001. Generally, in the case of the prodrug, the inhibitory activity is expected to be absent in vitro, as the prodrug requires the presence of plasma enzymes to hydrolyse the prodrug moiety and allow the formation of the parent active compound. However, in our primary in vitro assay, blood is a component of the assay and is used to assess the inhibition of haemolysis induced by pneumolysin. Therefore, we observe some inhibitory activity in the presence of the prodrug UL7-002, due a certain degree of enzymatic cleavage of the prodrug moiety, occurring during the 40 minute incubation in blood, which leads to the release of the parent active compound UL7-001. In summary, this assay demonstrates the in vitro activity of the parent active compound UL7-001 and indicates that the prodrug converts to the parent active compound in the presence of blood. This conversion to the parent active compound is further demonstrated in Section F.

IC₅₀ values of examples shown in Table 1 are as follows: Parent active compound UL7-001: IC₅₀ 0.3 μM; Prodrug UL7-002: IC₅₀ 6.8 μM.

B. Solubility and Chemical Stability Testing for the Determination of a Suitable Formulation for Intravenous Administration

Rationale

Parenteral delivery is one preferred route of administration of compounds of the invention. Therefore, prodrug UL7-002 was designed to improve the solubility and chemical stability in aqueous buffers of the parent active compound UL7-001, in order to achieve a readily soluble formulation, with enhanced chemical stability that could be reconstituted at the bed side, at a high concentrations, in safe saline solutions, compatible with intravenous administration.

Experimental procedure

Solubility Testing

Solubility studies were performed by charging a vial with 5-10 mg of compound followed by the addition of PBS solution to achieve a concentration of 100 mg/ml. If solubility was not observed, the solution was diluted to concentrations of 50 mg/ml, 25 mg/ml and 4 mg/ml consecutively until complete solubility was observed.

Chemical Stability Assessment

Stability studies were performed by dissolving 1-2 mg of compound in DMSO (1 ml) followed by addition of 0.4 ml of the resulting solution to stirred PBS (9.6 ml) at 37.5° C. A sample (-j 0.5 ml) was immediately taken for HPLC analysis. Further samples were then taken for analysis at various time-points thereafter. Half-lives were determined from the decrease in concentration of compound with respect to time.

Results

The formulations obtained with Examples UL7-001 and UL7-002 are shown in Table 2. Both compounds proved to be readily soluble in aqueous formulations that are compatible with safe intravenous dosing at the desired concentrations. In addition, the prodrug UL7-002, exhibited an improved chemical stability in aqueous formulations, in relation to the parent active compound UL7-001, with a t_{1/2 2} of 47 h.

TABLE 2
Properties of the formulations of compounds of the invention

| | | Chemical stability in |
|---|---|---|
| Example | Solubility in PBS* pH 7.2 | aqueous formulation (t½) |
| UL7-001 | Soluble at 100 mg/ml | <30 min. |
| UL7-002 | Soluble at 100 mg/ml | 47 h |

*PBS: Phosphate Buffered Saline

C. Secondary In Vitro Assay: Inhibition of Pneumolysin-Induced Lactate Dehydrogenase Release

Rationale

Pneumolysin induces the release of lactate dehydrogenase (LDH) from human monocytes and lung epithelial cells: a phenomenon that is indicative of plasma membrane damage or rupture [Infect. Immun. (2002) 70 1017-1022]. The LDH assay may be applied to demonstrate the ability of the disclosed compounds to inhibit the cytotoxic effect of pneumolysin on human lung epithelial cells in culture. The use of this assay can provide two main pieces of information on (1) Activity, to demonstrate the inhibition of LDH release from cells exposed to pneumolysin in the presence of inhibitory compounds versus the LDH release from cells exposed to pneumolysin alone, (2) Compound toxicity, the assay format is designed so it allows, in the control wells, the testing of the LDH release from cells exposed to the compound only.

Experimental Procedure

Human lung epithelial cells (A549) are seeded in flat-bottomed 96-well tissue culture plates and grown in RPMI 1640 medium supplemented with Glutamine, at 37° C., 5% CO₂, for 24 h. Before use, the cells are washed with PBS. Test compound dilutions are incubated with pneumolysin as described in Section A, then transferred to wells containing the human lung epithelial cells and the plates are incubated at 37° C., 5% CO₂, for 30 min. The following controls are included on the plate (1) Negative controls, called low control (PBS only) to measure the natural release of LDH from the cells in culture, (2) positive controls (1% (v/v) Triton-X in PBS) to measure the maximum release of LDH from the cells (3) Pneumolysin solution only to measure pneumolysin-induced LDH release, (4) Test compound solution to assess the toxicity of the compound alone. After incubation, the supernatant is transferred to the wells of round-bottomed 96-well microtitre plates containing a double volume of lactate dehydrogenase assay mixture (TOX7, Sigma) prepared according to manufacturer's instructions. Incubation in a light-proof chamber at RT for 5-10 min is followed by the addition of 1N HCl to all wells. Absorbance at 490 nm and 655 nm was then measured. The percentage of LDH release induced by pneumolysin in the presence and absence of test compounds is plotted against the Log of the concentration of the compound and the IC₅₀ is determined, as described above in the inhibition of haemolysis assay, Section A.

D. Ex Vivo Assay: Inhibition of the Effect of Pneumolysin on the Ciliary Function of Cultured Ependymal Cells

Rationale

The ependymal ciliated cells line the cerebral ventricles of the brain and the central canal of the spinal cord and are covered with cilia responsible for the circulation of the cerebrospinal fluid (CSF) around the central nervous system. This layer acts as a selective brain barrier to and from the cerebrospinal fluid and plays a role in controlling the CSF volume. To study if the inhibitors prevent the damage caused by pneumolysin on the ependymal layer, a rat ex vivo model of meningitis may be used. This model is based on culturing and differentiating ciliated ependymal cells from neonate rat brains, which recreate the in vivo situation, where cells lining the brain ventricles, are exposed to S. pneumoniae and its toxic products.

The use of the ex vivo model of meningitis constitutes a powerful means to predict the ability of a compound to prevent pneumolysin from causing damage in vivo.

Experimental Procedure

Ependymal cell cultures are prepared by the method previously described [Microb. Pathog. (1999) 27 303-309]. Tissue culture trays are coated with bovine fibronectin and incubated at 37° C. in 5% (v/v) CO₂ for 2 h before use. The growth medium is minimum essential medium (MEM) with added penicillin (100 IU/mL), streptomycin (100 μg/mL), fungizone (2.5 μg/mL), BSA (5 μg/mL), insulin (5 μg/ml), transferrin (10 μg/mL) and selenium (5 μg/mL). Neo-natal (0-1 day old) rats are killed by cervical dislocation, and their brains are removed. The cerebellum is removed along with edge regions of the left and right cortical hemispheres and the frontal cortex. The remaining brain areas are mechanically dissociated in 4 mL of growth medium. The dissociated tissue from one or two brains is added to the wells of the tissue culture trays (500 μl/well), each containing 2.5 mL of growth medium. The cells then are incubated at 37° C. in 5% (v/v) CO₂. The medium is replaced after three days and thereafter the ependymal cells are fed every two days with 2 mL of fresh growth medium supplemented with thrombin.

After approximately two weeks, the cells are fully ciliated and ready for experiments. To perform the experiments, the growth medium is replaced with 1 mL of medium MEM containing 25 mM HEPES, pH 7.4. The tissue culture trays are placed inside a thermostatically controlled incubation chamber surrounding the stage of an inverted light microscope. The cell cultures are allowed to equilibrate until the temperature of the assay medium was 37° C. At this point, recombinant purified pneumolysin, with and without test compound, pre-incubated in 1 ml of medium MEM at 37° C. for 40 min, is added to the wells containing the ciliated cells. To the control cells, 1 mL of MEM medium is added. Beating cilia are recorded before and after exposure over 30 min, with a digital high-speed video camera at a rate of 500 frames/s. The recorded video sequences are played back at reduced frame rates and the ciliary beat frequency (CBF) is determined by the following equation:

$$CBF ( Hz ) = \frac{500 frames \text{/} s}{( frames elapsed for 5 ciliary beat cycles )} \times 5 ( conversion per beat cycle ) .$$

E. In Vivo Efficacy Assay Using a Mouse Pneumonia Model

Rationale

This model has been well established in the laboratory of the inventors and has become adapted by other research groups working in this field. Using this model, pneumolysin was shown to be essential for the pathogenesis of S. pneumoniae and for its survival in vivo. With this disease model, mice infected with a strain of S. pneumoniae mutant deficient in pneumolysin (PLN-A), exhibited (1) a significant increase in the survival, (2) significant delay and attenuation of the signs of the disease and (3) substantial decrease in the pulmonary inflammation and less bacteraemia (infiltration of the bacteria from the lungs to the circulation). Therefore, this in vivo disease model constitutes a powerful tool to study the disease progression of mice infected with wild-type S. pneumoniae and treated with pneumolysin inhibitors. Survival is used as an endpoint parameter for the study.

Experimental Procedures: Infection, Treatment and Disease Signs Scoring

Outbred MF1 female mice, 8 weeks old or more and weighing 25-30 g are used. The animals are maintained under controlled conditions of temperature, humidity and day length. They have free access to tap water and pelleted food. The in vivo experiments are performed using two control groups: Control 1 (infected and not treated), Control 2 (not infected and treated) and one Treatment group (infected and treated). Mice in control group 1 and in the treatment group are infected intranasally with Streptococcus pneumoniae strain D39 (procedure described below). After completing the infections, the viable count of the given dose is determined (as described below). Subsequently, every six hours, animals in the treatment group and in the control group 2, receive the test compound intravenously, while excipient alone is administered to control group 1. The progress of the signs of disease (Table 3) is assessed every 6 h based on the scheme of Morton and Griffiths [Veterinary Record. (1985) 111, 431-436]. Animals are killed if they became 2+ lethargic and the time is recorded. The survival rates of control and test groups are compared with a log-rank test.

TABLE 3
Scoring scheme of the disease signs

| Sign | Description |
|---|---|
| Normal | Healthy appearance. |
| | Highly active. |
| 1+/2+ Hunched | Slight (1+) or pronounced (2+) |
| | convex curvature of the upper spine. |
| 1+/2+ Starey coat | Slight (1+) or pronounced (2+) |
| (Piloerection) | piloerection of the coat. |
| 1+/2+ Lethargic | Pronounced hunching and |
| | piloerection accompanied by a |
| | considerable (1+) or severe (2+) |
| | reduction of activity. |

The procedures which may be used for infection with S. pneumoniae, the delivery of the treatment and for the determination of the bacterial viable counts, mentioned above, are detailed as follows:

Intranasal Instillation of Infection

Mice are lightly anaesthetised with 2.5% (v/v) isoflurane over 1.6-1.8 L O₂/min. The confirmation of effective anaesthesia is made by observation of no pedal reflex. A mouse is held by the scruff of the neck in a vertical position with its nose upward. The infectious dose is then administered in sterile PBS, given drop by drop into the nostrils, allowing the animal to inhale it in between drops. Once the dose is given, the mouse is returned to its cage, placed on its back to recover from the effects of anaesthetic.

Intravenous Administration of Treatment

Mice are placed inside an incubator at 37° C., for 10 min, to dilate their veins. Each mouse is then individually placed inside a restrainer, leaving the tail of the animal exposed. The tail is disinfected with antimicrobial wipes. The treatment with the drug is administered intravenously every 6 h using a 0.5 ml insulin syringe inserted carefully into one of the tail lateral veins. Doses are prepared freshly and administered intravenously to the animals.

Determination of Viable Count of the Infectious Dose

Viable counting is performed by the method of Miles and Misra [J. Hyg. (1938) 38 732-749). 20 μL of the sample are serially diluted in 180 μL PBS in round-bottomed 96-wells microtitre plates, up to a dilution of 10⁶. Blood agar plates are divided into six sectors and 60 μL of each dilution plated onto an individual sector. The plates are incubated in CO₂ gas jars overnight at 37° C. The following day, colonies are counted in the sector where 30-300 colonies are visible. The concentration of colony forming units (CFU) per millilitre is determined by using the following equation:

$$CFU per ml = \frac{Number of colonies in sector}{60 µl} \times Dilution \times 1000 ( conversion factor ) .$$

F. Conversion of Prodrug UL7-002 to Active Inhibitors in Mouse Plasma

Rationale

To demonstrate that the prodrug is converted to the parent active compound in the presence of plasma enzymes, the prodrug derivative was incubated with mouse plasma at 37° C. at 5 time points over a 2 h period. The samples were then analysed by LC-MS/MS to obtain the amount of active compound appearing and prodrug derivative remaining over time.

Experimental Procedure

The prodrug derivative of the invention was assessed in mouse plasma stability assay at a concentration of 10 μM. Test compounds were diluted in DMSO to a final stock concentration of 10 mM. For the purpose of the assay, the stocks prepared were further diluted in DMSO to a concentration of 400 μM and 5 μL were added to 195 μL of mouse plasma (pH 7.4) and then incubated at 37° C. The final concentration of DMSO in the plate was 2.5% (v/v). Reactions were terminated at 0, 15, 30, 60 and 120 min after incubation by adding 400 μL of acetonitrile containing 0.55 μM metoprolol and 1% (v/v) formic acid. The plate was then centrifuged at 3000 rpm, for 45 min, at 4° C. 80 μL of supernatant were transferred into a conical bottom 96 well glass coated plate. 40 μL of water were added prior to analysis for prodrug derivative and active species by LC-MS/MS. This assay was performed by a contract research organisation, Cyprotex Discovery Limited, UK, at the request of the inventors at Leicester.

Results

The quantification of the prodrug compound remaining and the parent active compound appearing was performed as follows:

(1) The parent active compound was quantified using a 6 point calibration curve prepared in deactivated mouse plasma. (2) The percentage of prodrug compound remaining at each time point relative to 0 min sample was calculated from LC-MS/MS peak area ratios (compound peak area/internal standard peak area). This percentage was then used to determine the concentration of the prodrug compound at each time point in reference to the starting concentration (10 μM) at time 0 min. The conversion of the prodrug UL7-002 to its parent active compound UL7-001 is shown in Table 4.

### CONCLUSION

The results presented in Table 4 clearly indicate the therapeutic benefits of the prodrug of the invention, which is demonstrated by its rapid conversion in plasma into the parent active compound. Besides the therapeutic benefit, the physicochemical properties of UL7-002 are favourable for the preparation of formulations suitable for parenteral delivery.

TABLE 4

| Prodrug | Prodrug/ | [ ] (μM) | [ ] (μM) | [ ] (μM) | [ ] (μM) | [ ] (μM) |
|---|---|---|---|---|---|---|
| ID | Active* | t_{0 min.} | t_{15 min.} | t_{30 min.} | t_{60 min.} | t_{120 min.} |
| UL7-002 | Prodrug | 10.00 | 0.00 | 0.01 | 0.00 | 0.00 |
| | (Mouse) | | | | | |
| | (UL7-002) | | | | | |
| | Active | 0.17 | 10.00 | 9.60 | 10.33 | 11.56 |
| | (Mouse) | | | | | |
| | (UL7-001) | | | | | |

Throughout the specification and the claims which follow, unless the context requires otherwise, the word ‘comprise’, and variations such as ‘comprises’ and ‘comprising’, will be understood to imply the inclusion of a stated integer, step, group of integers or group of steps but not to the exclusion of any other integer, step, group of integers or group of steps.

All patents and patent applications referred to herein are incorporated by reference in their entirety.

The application of which this description and claims forms part may be used as a basis for priority in respect of any subsequent application. The claims of such subsequent application may be directed to any feature or combination of features described herein. They may take the form of product, composition, process, or use claims and may include, by way of example and without limitation, the claims.

1. A compound of formula (I): 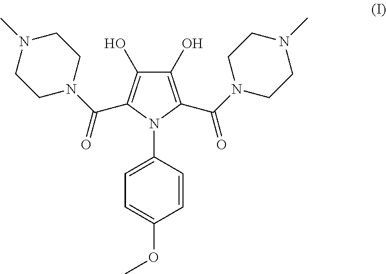
or a pharmaceutically acceptable prodrug derivative thereof, or a pharmaceutically acceptable salt or solvate thereof.

2. A compound according to claim 1 in the form of a prodrug derivative.

3. A compound according to claim 2 wherein the prodrug derivative is selected from carboxylate ester, sulfamate ester, phosphate ester and carbamate ester derivatives.

4. A compound according to claim 3 wherein the prodrug derivative is a carboxylate ester derivative.

5. A compound according to claim 3 of formula (Ia): 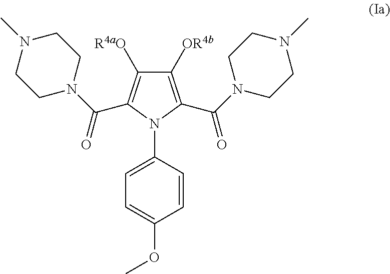
wherein one or both of R^{4a} and R^{4b} are independently selected from —C(O)R¹⁶, —SO₂NH₂, —PO(OR¹⁹)(OR²⁰), —CHR²⁶—OPO(OR¹⁹)(OR²⁰) where R²⁶ is hydrogen or C₁-C₆ alkyl, and —C(O)NR¹⁷R¹⁸, wherein R¹⁶, R¹⁷, R¹⁸, R¹⁹ and R²⁰ are independently selected from:
(a) C₁-C₆ alkyl, C₂-C₆ alkenyl, C₂-C₆ alkynyl, C₃-C₁₀ cycloalkyl, C₅-C₁₀ cycloalkenyl, heterocyclyl, —C₁-C₃ alkyl-C₃-C₁₀ cycloalkyl, —C₁-C₃ alkyl-C₅-C₁₀ cycloalkenyl or —C₁-C₃ alkylheterocyclyl, or R¹⁷ and R¹⁸ together with the N to which they are attached may form a 5- or 6-membered heterocyclic ring optionally containing a further heteroatom selected from O, S and NR^{25a}R^{25b} where R^{25a} is hydrogen, C₁-C₆ alkyl, —CH₂—OPO(OR¹⁹)(OR²⁰) or a 5- or 6-membered heterocyclic ring, and R^{25b} is absent or C₁-C₆ alkyl; and in which any of the aforementioned R¹⁶, R¹⁷ or R¹⁸ groups may be optionally substituted by one or more groups selected from cyano, —OPO(OR¹⁹)(OR²⁰), —(O(CH₂)_{z})ᵣOR²⁴, wherein each z, which may be the same or different, represents 2 or 3, r represents an integer selected from 1 to 20, and R²⁴ is hydrogen, C₁-C₃ alkyl or —PO(OR¹⁹)(OR²⁰), C₁-C₆ alkoxy, C₁-C₆ fluoroalkoxy, C₁-C₆ alkyl, C₁-C₆ fluoroalkyl and —C(O)NR^{a}R^{b}, where R^{a} and R^{b} are independently selected from hydrogen and C₁-C₆ alkyl, and any of the aforementioned R¹⁶, R¹⁷ or R¹⁸ groups may be optionally substituted by one or more halogen atoms; and
(b) aryl, heteroaryl, C₁-C₃ alkylaryl and —C₁-C₃ alkylheteroaryl, said aryl and heteroaryl groups being optionally substituted;
or R¹⁸, R¹⁹ and R²⁰ may independently represent hydrogen.

6. A compound according to claim 5 wherein one or both of R^{4a} and R^{4b} are independently selected from —C(O)R¹⁶, —SO₂NH₂, —PO(OR¹⁹)(OR²⁰) and —C(O)NR¹⁷R¹⁸, wherein R¹⁶, R¹⁷, R¹⁸, R¹⁹ and R²⁰ are independently selected from
(a) C₁-C₆ alkyl, C₂-C₆ alkenyl, C₂-C₆ alkynyl, C₃-C₁₀ cycloalkyl, C₅-C₁₀ cycloalkenyl, heterocyclyl, —C₁-C₃ alkyl-C₃-C₁₀ cycloalkyl, —C₁-C₃ alkyl-C₅-C₁₀ cycloalkenyl or —C₁-C₃ alkylheterocyclyl, in which any of the aforementioned R¹⁶, R¹⁷ or R¹⁸ groups may be optionally substituted by a group selected from cyano, C₁-C₆ alkoxy, C₁-C₆ fluoroalkoxy, C₁-C₆ alkyl, C₁-C₆ fluoroalkyl and —C(O)NR^{a}R^{b}, where R^{a} and R^{b} are independently selected from hydrogen and C₁-C₆ alkyl, and any of the aforementioned R¹⁶, R¹⁷ or R¹⁸ groups groups may be optionally substituted by one or more halogen atoms, and
(b) aryl, heteroaryl, C₁-C₃ alkylaryl and —C₁-C₃ alkylheteroaryl, said aryl and heteroaryl groups being optionally substituted;
or R¹⁸, R¹⁹ and R²⁰ may independently represent hydrogen;
and wherein when one of R^{4a} and R^{4b} are independently selected from the groups defined above the other is hydrogen.

7. A compound according to claim 5 wherein both of R^{4a} and R^{4b} are independently selected from —C(O)R¹⁶, —SO₂NH₂, —PO(OR¹⁹)(OR²⁰), —CHR²⁶—OPO(OR¹⁹)(OR²⁰ where R²⁶ is hydrogen or C₁-C₆ alkyl, and —C(O)NR¹⁷R¹⁸.

8. A compound according to claim 5 wherein one of R^{4a} and R^{4b} is selected from —C(O)R¹⁶, —SO₂NH₂, —PO(OR¹⁹)(OR²⁰), —CHR²⁶—OPO(OR¹⁹)(OR²⁰) where R²⁶ is hydrogen or C₁-C₆ alkyl, and —C(O)NR¹⁷R¹⁸; and the other of R^{4a} and R^{4b} is hydrogen.

9. A compound according to claim 5 wherein one or both of R^{4a} and R^{4b} are independently selected from —C(O)R¹⁶.

10. A compound according to claim 9 wherein R¹⁶ is C₁-C₆ alkyl or C₃-C₁₀ cycloalkyl in which either of the aforementioned groups may be optionally substituted by a group selected from —OPO(OR¹⁹)(OR²⁰) and —(O(CH₂)_{z})ᵣOR²⁴, where each z, which may be the same or different, represents 2 or 3, r represents an integer selected from 1 to 20, and R²⁴ is hydrogen, C₁-C₃ alkyl or —PO(OR¹⁹)(OR²⁰) or R¹⁶ is phenyl optionally substituted by —(CHR²⁶)_{q}—OPO(OR¹⁹)(OR²⁰) wherein q represents 0 or 1.

11. A compound according to claim 10 wherein R¹⁶ is C₁-C₆ alkyl.

12. A compound according to claim 11 wherein R^{4a} and R^{4b} are —C(O)CH(CH₃)₂.

13. A compound according to claim 1 which is a di-hydrochloride salt.

14. A pharmaceutical composition comprising a compound according to claim 1, optionally in combination with one or more pharmaceutically acceptable diluents or carriers.

15. A pharmaceutical composition according to claim 14 comprising one or more additional therapeutically active ingredients.

16. (canceled)

17. (canceled)

18. A method of treating bacterial infections caused by bacteria producing pore-forming toxins, such as cholesterol dependent cytolysins, in a subject, said method comprising administering to said subject a therapeutically effective amount of a compound according to claim 1.

19. The method according to claim 18 wherein the bacterial infection is caused by Streptococcus spp. (e.g. Streptococcus pneumoniae, Group A Streptococci or Streptococcus suis), Clostridium spp. (e.g. Clostridium perfringens), Listeria spp. (e.g. Listeria monocytogenes) or Bacillus spp. (e.g. Bacillus anthracis).

20. The method according to claim 19 for wherein the bacterial infection is caused by Streptococcus pneumoniae.

21. The method according to claim 20 for the treatment of pneumococcal pneumonia, pneumococcal meningitis, pneumococcal septicaemia/bacteraemia, pneumococcal keratitis or pneumococcal otitis media.

22. The method according to claim 18 for the treatment of a condition selected from gas gangrene, gastrointestinal anthrax, inhalational anthrax, porcine meningitis, encephalitis, septicaemia/bacteraemia and pneumonia which are caused by bacteria other than pneumococcus.

23. The method according to claim 16 wherein the compound is administered in combination with one or more additional therapeutically active ingredients (e.g. one or more antimicrobial or immunomodulatory agents).

24. (canceled)

25. A protected derivative of the compound of formula (I) according to claim 1.

26. A process for preparing the compound of formula (I) according to claim 1 which comprises reacting a compound of formula (II) or a protected derivative thereof: 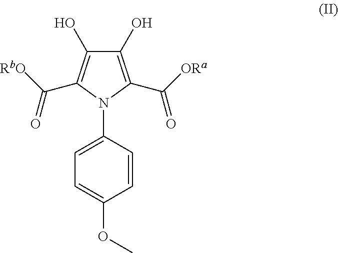
with 1-methylpiperazine and, if required, deprotecting the resulting compound.

27. A process for preparing the compound of formula (Ia) according to claim 5 which comprises reacting a compound of formula (I): 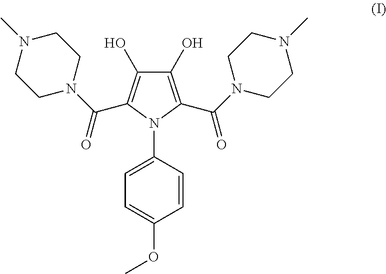
or a protected derivative thereof, with a compound of formula R^{4a}Z, a compound of formula R^{4b}X, or a combination thereof, where X is a leaving group.
